(19) United States
(12) Patent Application Publication
(10) Pub. No.: US 2005/0261582 A1
(43) Pub. Date: Nov. 24, 2005

(54) Intracorporeal probe with disposable probe body

(76) Inventors: Matthew Becker, Harrisburg, PA (US); Kevin Mack, Fleetwood, PA (US); Otto Gertler, Ephrata, PA (US); Mark Schafer, Ambler, PA (US); Rod Denlinger, Lancaster, PA (US); Angela Lassandro, Orwigsburg, PA (US); Stanley Kniezewski, Blue Bell, PA (US); Dana Randolph, Malden, MA (US); Jason Lassandro, Orwigsburg, PA (US); Bill Regan, Tewksbury, MA (US)

Correspondence Address: Karl J. Kolbinger;c/o Amster, Rothstein & Ebenstein LLP, 90 Park Avenue, New York, NY, 10016, US

(21) Appl. No.: 10/848,086

(22) Filed: May 18, 2004

(51) Int. Cl. A61B 8/00

(52) U.S. Cl. 600/437

(57) ABSTRACT

An ultrasonic probe for flow rate measurement is disclosed having a plurality of transducers mounted on a flexible cable configured to be inserted in a body cavity to obtain cardiac or other biometric telemetry. The flexible cable is provided with a disposable body which covers and microbially isolates the flexible cable and transducers. The disposable body is flexible and permits rotation of the flexible cable within it. Removal and replacement of the disposable body allows the probe to be used again without sterilization of the probe itself. A sensor window may be provided in the disposable body to allow transmission of ultrasonic signals, said window being provided with an anechoic surface treatment to reduce reception spurious ultrasonic echoes due to the material of the sensor window.

### FIELD OF THE INVENTION

The present invention relates to a flexible intracorporeal probe, and more particularly to a probe having a flexible torque transmitting sensor portion covered by a flexible body which can be removed for disposal and replacement between uses.

### BACKGROUND OF THE INVENTION

Hemodynamic monitoring is a useful and necessary tool in the management of critically ill patients which can present a wide variety of potential problems for clinicians which may result in an increase in mortality and morbidity of a patient. For example, with the development of the Swan Ganz Thermodilution Catheter (SGC, Edwards Laboratories, Irvine Calif.), many patients who were otherwise deemed too risky to have open heart surgery could undergo these grossly invasive procedures. However, use of the SGC presents its own risks to the patient.

The SGC is a multi-lumen catheter that is placed through the right heart by way of internal jugular, subclavian or femoral veins. Once placed by fluoroscopy, the practice became standard to float the SGC by means of monitoring pressure wave form changes as the SGC passed from the right atrium through the heart into the pulmonary artery trunk. With a balloon on the tip of the catheter, the blood flow pulls the tip through the heart easily with it “wedging” in a branch of the smaller pulmonary artery trunk. This enables clinicians to look at pressures behind and forward of the catheter tip, indirectly measuring pressures in the left atrium.

These measurements are important because it is widely believed that the wedge pressure is directly related to the filling process of the left heart. These changes are thought to indicate an indirect measurement of left heart function, thus enabling clinicians to treat a patient by titrating fluids and the appropriate vaso-active drugs to alter the function of the heart and peripheral vasculature.

Although the SGC was a major milestone in medical practice, widespread use of the SGC has led to increases in mortality and morbidity directly related to its invasiveness. A strong need existed for a device and method for assessing left heart function without the risks associated with a highly invasive procedure.

U.S. Pat. No. 5,479,928 to Cathignol et al. describes a less invasive intracorporeal ultrasonic probe for accurately determining the speed of a liquid medium, and in particular blood flow rate in the aorta.

Such a probe comprises an inner, sensor portion and an outer, body portion. The sensor portion comprises a flexible torque transmitting cable which is attached at one end to one or more ultrasonic transducers, and at the other end to a drive member. The sensor portion is disposed concentrically within a body or housing within which the sensor portion is free to rotate. Ideally, both the sensor and body portions of the probe are sufficiently flexible to permit the probe to be comfortably placed within the body of a patient, for example, within the esophagus.

When placed in this manner within a patient, the drive member may be rotated causing the sensor portion, and particularly the ultrasonic transducers, to be rotated azimuthally relative to the body portion, for the purpose of precisely aligning the ultrasonic transducers to the desired region of the body (e.g., to the precise area of the esophagus which is adjacent to the aorta). Signals from the transducers are transmitted by conductors in the flexible cable to the drive member where the signal from which the transducers are picked up for analysis by processing circuitry, usually a monitor connected by an interface cable to the sensor portion of the probe. In this way, an accurate measurement of cardiovascular parameters can be achieved using a minimally invasive probe.

A significant drawback of such a probe is the need for disinfection and/or sterilization prior to use on a patient. Such treatment is lengthy, expensive, and difficult to implement. It is necessary to apply agents which are expensive, conscientiously to the probe. Care must be taken of the probe while it is being handled in this way since such a probe is fragile, inherently flexible, and the agents can be harmful. In addition, it is necessary to repeat the process on each occasion that the probe is used, which is inconvenient and increases the risk of damaging the probe.

The problem of disinfection is partially addressed by U.S. Pat. No. 6,350,232 to Hascoet et al. which discloses a device for applying a thin, resilient jacket to the body of an ultrasonic intracorporeal probe, the jacket being discarded after each use. The jacket is a flexible tube made of a resilient material such as silicone or a natural synthetic rubber. The tube is closed to form a tip at one end which contains an impedance-matching medium, such as a gel for matching impedance between the disposable jacket and the measuring elements. The gel is necessary to displace any air which might disturb the transmission of the ultrasonic sound waves through the jacket and into the patient. The cover has a length sufficient to cover the outside surface of the probe, therefore a complex sterilization or disinfection procedure need not be performed on the body of the probe, as a new, presumably sterile probe jacket is applied to the probe prior to each use in the body of a patient.

The device for the application of the jacket to such a long flexible probe body results in a number of difficulties. For example, due to the typical length of an intracorporeal probe, particularly those intended for insertion in the esophagus, it has proven difficult in practice to place a thin, resilient jacket completely over the body of a probe without damaging the probe or the jacket.

Other difficulties are due to the nature of the jacket itself. For example, the insertion of the probe into the disposable jacket necessarily displaces any air that may be in the jacket. However, due to its resiliency, the jacket tends to seal itself against the probe body leading to a build-up of air in the tip of the jacket. The air resists further insertion of the probe into the jacket, and may rupture the jacket compromising the cleanliness of the probe. Furthermore, any air that migrates to the proximity of the ultrasonic transducers may impede the transmission of the ultrasonic signal.

Thus, the device disclosed by Hascoet et al. provides a complex, vacuum-driven mechanism to support the jacket during insertion of the probe and to cause the jacket to expand to a diameter sufficient to permit the escape of displaced air. After the probe body is fully inserted into the jacket, it is released from the device. The device is large and unwieldy and significantly complicates the process of preparing the probe for use. Furthermore, however thin the jacket may be, it imposes a layer of material between the transducers and the target blood vessel which can attenuate the signal and/or result in undesirable signal “ghosting”.

A further drawback of the prior art flexible jacket is that there is no effective method for confirming that the jacket has been completely installed over the probe body, and that the jacket will not migrate off of the probe in situ, potentially exposing the patient to non-sterile portions of the probe.

A still further drawback of the prior art probe is that the drive member is free to rotate the sensor portion relative to the body of the probe through multiple revolutions in the same direction, limited only by the physical limitations presented by the interface cable between the probe and its circuitry. Ultimately, repeated twisting of the interface cable to its physical limits can result in premature failure of the interface cable.

Therefore, a need exists for a flexible intracorporeal probe which can be readily prepared for hygienic use in a patient without the need for the application of conventional disinfectants.

A further need exists for a flexible intracorporeal probe having a disposable microbial barrier that can be applied without the need for a complicated applicator.

A still further need exists for a microbial barrier for a flexible ultrasonic intracorporeal probe which minimizes the attenuation and/or disruption of the ultrasonic sensing signals generated by the probe.

A still further need exists for a flexible intracorporeal probe which can provide a positive confirmation that the microbial barrier has been completely installed.

A still further need exists for a flexible intracorporeal probe having a sensor portion which is internally limited to prevent the sensor portion from rotating relative to its body portion beyond a predetermined range of motion.

### SUMMARY OF THE INVENTION

The invention seeks to resolve these problems and satisfy these needs by proposing a flexible intracorporeal probe having a flexible body portion which is disposable and provides a microbial barrier to prevent non-sterile portions of the probe from contact with the patient, thus eliminating the need for comprehensive sterilization or disinfection of the probe surfaces, or a separate disposable jacket to cover the body portion of the probe.

The disposable flexible body portion is formed of a material which is sufficiently rigid to receive the sensor portion of the probe without the need for a separate applicator. Thus, the invention guarantees hygiene for the patient during use of the probe without the need for a separate disposable flexible jacket. Indeed, the disposable body of the present invention serves both the function of the body portion of the prior art probe, and the antimicrobial function of the protective jacket, thereby eliminating the need for the latter component and simplifying assembly and preparation of the probe for use on a patient.

In a preferred embodiment, a section is provided in the disposable probe body which is transparent to sensing signals produced by the sensing elements of the probe. This “sensor window” is positioned on the body in a location that corresponds, when assembled, to that of the sensing elements in the sensor portion of the probe. The sensor window minimizes attenuation of the sensing signal, and may advantageously be provided with features to reduce or eliminate the reception of spurious signals which might otherwise interfere with the accurate measurement of cardiovascular parameters in the patient.

In another preferred embodiment, the probe of the present invention also provides a means for determining when the body portion has been completely installed, to insure the integrity of the microbial barrier feature prior to use of the probe. The means may include a light or other indicator which illuminates upon completion of the seal.

It is another object of the present invention to provide an internal limit to the rotation of the sensor portion relative to the body of the probe to minimize strain on any interface cable that may be needed between the probe and supporting circuitry.

In accordance with an embodiment of the invention, the sensor portion of the probe comprises a torque cable composed of a plurality of opposing coil layers preferably encapsulated in a flexible material such as polyurethane. The torque cable has a plurality of sensing elements, preferably ultrasonic transducers disposed at a distal end thereof, the proximal end of the torque cable attached to a handle or base. The disposable body is preferably an extruded tube composed of an inner layer of fluoropolymer such as FEP and outer layer of a flexible material such as polyurethane which encapsulates a reinforcing member such as fiber winding, or braid which may be composed of an aramid fiber. Also advantageously, a metal wire is suitable for this purpose.

Advantageously, the closed tip of the body forms the sensor window and is preferably molded from a clear material such as polyurethane. The tip may be heat bonded to the distal end of the disposable body, and may have a ribbed inner surface. The proximal end of the disposable body is advantageously provided with an annular hub which is preferably made of a rigid plastic material which advantageously is molded to the proximal end of the disposable body.

The construction of the disposable body allows it to retain its tubular configuration without the assistance of a rigid applicator, while remaining sufficiently flexible to accommodate the range of motion of the sensor portion of the probe. Due to its rigid tubular construction, the disposable body can be placed over the sensor portion of the probe easily and without the assistance of an applicator.

Preferably, a portion of the disposable body, advantageously the tip which comprises the sensor window, is filled with an impedance matching medium, in particular a gel for matching impedance between the body and the measuring elements of the sensor. The self supporting nature of the disposable body permits the escape of air due to displacement during insertion of the sensor portion of the probe into the body, as a small gap between the outer surface of the sensor portion and the inner surface of the disposable body is maintained.

A preferred embodiment of the invention also provides a connector in the base of the probe which receives the hub of the disposable body. The base of the probe may also be provided with a sensor such as a mechanical or optical switch which can detect the proper placement of the hub in the base and can provide visual confirmation to the user, such as by an indicator light, that the body has been properly installed and therefore that a reliable microbial barrier has been established between the sensor portion of the probe and the patient.

A further embodiment of the present invention provides a base attached to the proximal end of the sensor portion of the probe, said base having a flange mounted thereon which receives the hub of the disposable body. In a preferred embodiment, the base and the flange are rotateably attached to each other, such that the base can be rotated relative to the flange through a limited range of motion such as 540°, said rotation resulting in a corresponding displacement between the sensor portion of the probe and its body.

According to a further aspect of the present invention, a protective tip cap is provided for preventing the proximal migration of the impedance matching medium, or acoustic gel out of the tip. The tip cap may advantageously comprise a compressible, resilient sleeve, formed for example of synthetic rubber, which is selectively compressed by a rigid plastic structure around the probe body proximal to said tip, or sensor window. Preferably, the resilient sleeve is configured to distribute the compression along the entire surface of contact between the probe body and the sleeve, reducing the risk of damage to the sleeve as a result of the compression. Furthermore, the tip cap may comprise a closed tip housing dimensioned to prevent rupture of the tip due to expansion. A hole in the tip housing may also be advantageously employed to permit ingress of antiseptic agents during sterilization.

### BRIEF DESCRIPTION OF THE DRAWINGS

FIG. 1a is a side view of the probe of an embodiment of the present invention with the probe body removed.

FIG. 1b is a cross-sectional view of the probe of FIG. 1a

FIG. 2a is a side view of the uncoated sensor cable of an embodiment of the present invention.

FIG. 2b is a cross-sectional view of the uncoated sensor cable of an embodiment of the present invention revealing the structure thereof.

FIG. 3a is a side view of the probe body of an embodiment of the present invention.

FIG. 3b is a side and perspective detail showing the body tip of an embodiment of the probe body.

FIG. 3c is a side and perspective detail showing the hub of an embodiment of the probe body.

FIG. 4a is a side view of the plug rod assembly of an embodiment of the present invention.

FIG. 4b is a perspective view of the plug rod assembly partially inserted within the probe body of an embodiment of the present invention.

FIG. 5a is a graph illustrating the signal profile of the probe of an embodiment of the present invention.

FIG. 5b is a graph illustrating the signal profile of a modified embodiment of the probe.

FIG. 6a is a perspective view of an embodiment of a tip cap of the present invention.

FIG. 6b is a cross sectional view of the tip cap of FIG. 6a.

### DETAILED DESCRIPTION OF THE PREFERRED EMBODIMENTS

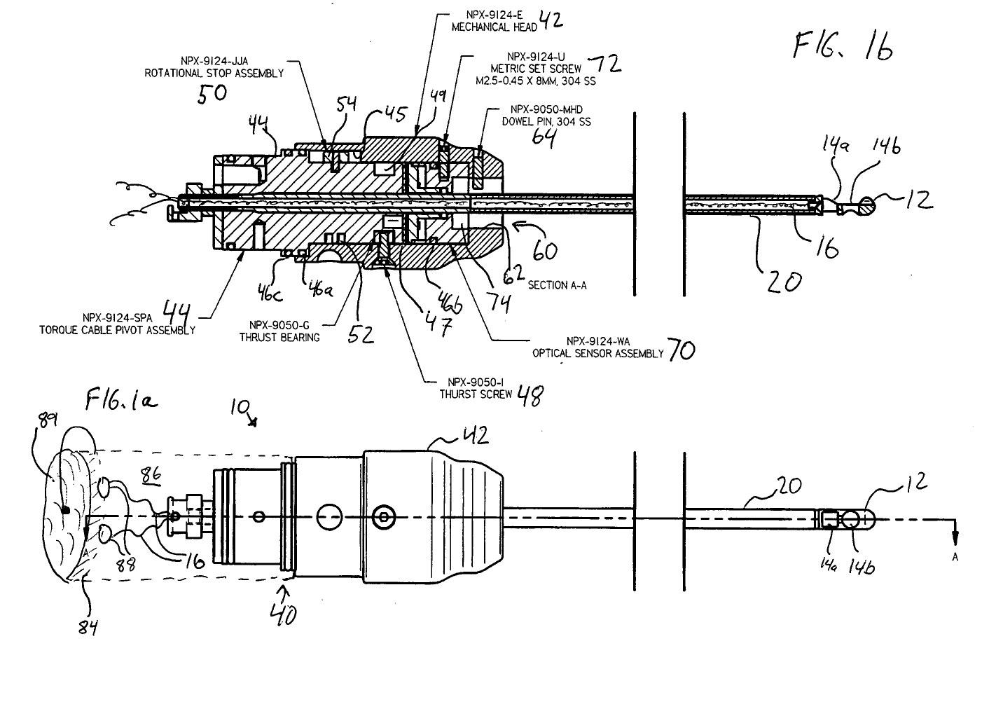

As shown in FIGS. 1a and 1b, an embodiment of probe 10 of the present invention is shown. Transducer cage 12 is at the distal end of probe 10 and is connected via sensor cable 20 to base 40 at the proximal end of probe 10.

Transducer cage 12 is preferably metallic and has a generally bullet-shaped cylindrical profile preferably having a cross sectional diameter of about 5 millimeter (mm) or less, which houses sensing elements 14a and 14b. Transducer cage 12 may also be provided with one or more grooves to facilitate distribution of ultrasonic gel over sensing elements 14. When probe 10 is to be used in place of a Swan Ganz Catheter for monitoring left ventricular function, the sensing elements are preferably crystal ultrasonic transducers, with sensing element 14a arranged at an angle which is offset 60 degrees relative to the angle of sensing element 14b. Transducer conductors 16 respectively carry electromagnetic signals to and from the sensing elements 14a and 14b, and are ideally connected to supporting circuitry, discussed below, to provide cardiovascular telemetry during use. The precise type, number and orientation of sensing elements in the probe of the present invention may be varied as known by those skilled in the art to perform a variety of monitoring functions. Thus, the structure of the cage may be provided with variously angled mounts for various sensing elements 14 as called for by the particular use for which it is employed.

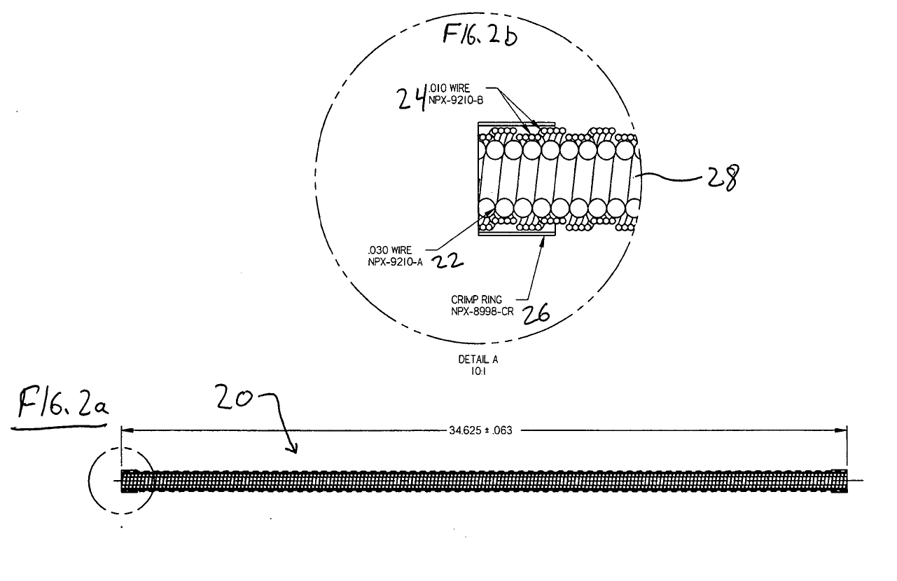

Sensor cable 20 is a flexible, torque-transmitting driveshaft. Sensor cable 20 is preferably narrow, having a radius of about 5 mm or less, and may be formed of a plurality of concentric springs. Ideally, sensor cable 20 has the same diameter as transducer cage 12 to permit the surface of the transducer cage to be approximately flush at its connection to sensor cable 20. The springs that form the structure of sensor cable 20 may advantageously be formed of a plurality of round surgical stainless steel wires having a high Young's modulus and a plurality of diameters. For example, as shown in FIGS. 2a and 2b, an inner winding 22 of larger diameter wire, such as 0.030 inch diameter round, medical grade 316 LVM, stainless steel is formed, for example, by winding clockwise about a round core wire (not shown), which is also ideally stainless steel having a uniform diameter such as 0.063 inches. Outer windings 24 may then be formed over inner winding 22 using a 0.010 inch diameter round, medical grade 316 LVM stainless steel wire, applied with an alternating bias. Crimp ring 26 is advantageously provided at each end of sensor cable 20 to secure the windings, preventing them from unwrapping. The length of sensor cable 20 may vary, with 65 centimeters or 85 centimeters being typical. The removal of the core wire from sensor cable 20 after winding provides coaxial channel 28 which extends along the length of sensor cable 20 from its proximal to its distal end, and provides a conduit for transducer conductors 16.

Sensor cable 20 is preferably coated in polyurethane or a similar elastomer for example, by dipping, extrusion or heat shrinking, which serves to encapsulate the three windings that comprise sensor cable 20. Additionally, one or more strands of high-strength fiber, such as Kevlar (not shown) may be provided along the length of sensor cable 20. Ideally, the fiber should be flexible, but relatively inelastic, serving to prevent any longitudinal distortion of the sensor cable, and increasing its tensile strength, without significantly reducing its flexibility. Ideally, neither the polyurethane coating nor the strands of fiber are present in coaxial channel 28 in sufficient quantities to block coaxial channel 28 at any point along the length of sensor cable 20.

Although the particular design of sensor cable 20 set forth above provides a connection between probe base 40 and transducer cage 12 which is both longitudinally flexible and angularly rigid, other torque cable designs known to those of skill in the art to provide the same characteristics may also be employed. Furthermore, it is preferable that sensor cable 20 have the same characteristics of angular rigidity when transmitting torque in either the clockwise counterclockwise direction, and that such angular rigidity remains generally constant whether or not sensor cable 20 is undergoing flexion.

Referring again to FIGS. 1a and 1b, probe base 40 is shown to be comprised of two subparts, body base 42 and sensor base 44, each having a proximal and a distal end. Sensor base 44 is attached at its distal end to the proximal end of sensor cable 20 and is shown concentrically journalled within bore 47 of body base 42 to permit rotation of sensor base 44 relative to body base 42. Bore 47 extends axially through body base 42 from its proximal end, where bore 47 is preferably of a sufficient radius to admit the distal end of sensor base 44 and wherein the radius of bore 47 is at least sufficient to admit the passage of sensor cable 20 therethrough.

Thrust screw 48 extends radially through body base 42 into bore 47 and engages annular groove 49 in sensor base 44, preventing axial movement of body base 42 relative thereto without limiting relative rotation of the subparts 42 and 44.

Preferably, a rotational stop assembly 50 links sensor base 44 to body base 42. Specifically, a preferred embodiment provides rotational stop assembly 50 comprising limiting thread 52 which is provided in sensor base 44 and a thread follower 54 which is disposed within slot 45 provided in bore 47 of body base 42 to limit the axial movement of the thread follower 54.

During rotation of sensor base 44 relative to body base 42, the corresponding rotation of thread 47 produces axial movement in thread follower 54, either toward the proximal or distal end of the probe, depending upon the thread and the direction of the rotation. When rotation in either direction results in the axial movement of thread follower 54 to the limits defined by slot 45, further rotation of sensor base 44 relative to body base 42 is prevented. The amount of rotation permitted by rotational stop assembly 50 depends entirely upon the pitch of limiting thread 52 and the amount of axial movement permitted by slot 45. Other means of limiting relative rotation of the subparts of base 40 will be known to a person of skill in the art which advantageously accomplish the same result as rotational stop assembly 50. A rotational limitation of more than one complete revolution such as about 540 degrees is ideal.

Body base 42 is preferably provided with a hub receptacle 60 which comprises annular shoulder 62 provided in bore 47 and a guide pin 64 which extends radially inward from annular shoulder 62. Optical sensor assembly 70 is provided within bore 47 and is held in place relative to body base 42 by set screw 72. Optical sensor assembly 70 is associated with hub receptacle 60 and contains sensor circuitry 74, either mechanical, electronic, or a combination thereof, well known in the art to detect the physical presence of a hub (discussed below) within hub receptacle 60. Optical sensor assembly 70 may provide confirmation of hub detection, for example, in the form of an audible or visual cue, which indicates that a hub has properly been inserted within hub receptacle 60.

O-rings 46a and 46b are shown recessed within an annular grooves in sensor base 44, and in sealing contact with bore 47. O-rings 46a and 46b may be provided to prevent the ingress of contaminants into bore 47 either from the proximal or distal ends respectively of body base 42, while allowing rotation of body base 42 and sensor base 44 relative to each other.

Sensor base handle 80 may be affixed to sensor base 44. Sensor base handle 80 ideally has about the same radial dimension as that of body base 42, and has a bore 82 and a closed end 84 which provides a hollow chamber 86 proximal to body base 42. O-ring 46c may be provided to prevent ingress of contamination into chamber 86. Ideally, one or more connectors 88 are provided in sensor base 80 to provide a terminus for transducer conductors 16. Alternately, probe circuitry (not shown) may be housed in chamber 86, said transducer conductors 16 connected to the circuitry. Further, a brush and commutator arrangement (not shown) may be employed to conduct transducer signals from sensing elements 14 to connectors 88 or to probe circuitry. A connector cover 89 may be provided on the proximal end of sensor base handle 80 to protect connectors 88 from the ingress of contaminants.

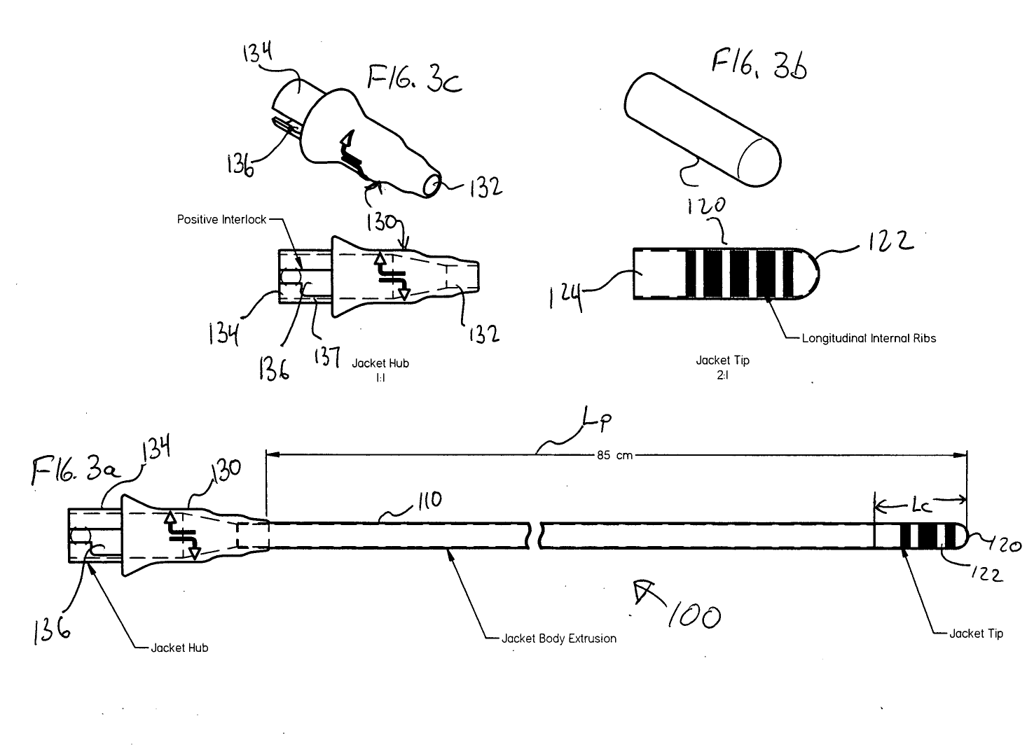

Viewed in conjunction with FIGS. 1a and 1b, FIG. 3 is a side view of probe body 100. The probe body comprises a hollow tube portion 110, having a body tip 120 at its distal end and a hub 130 at its proximal end.

Body tip 120 is preferably generally cylindrical, is preferably molded from a clear flexible material such as Texin®, manufactured by Bayer MaterialScience of Pittsburgh, Pa. which must be at least translucent, and is preferably transparent to the signals transmitted and detected by sensing elements 14. Body tip 120 has a closed end 122 at a distal end thereof and an opening 124 at a proximal end thereof, opening 124 having a diameter which is preferably slightly larger than the outer diameter of sensor cable 20 and is bonded, such as by heat to the distal end of the tube portion 110. The longitudinal dimension Lc of body tip 120 is preferably about equal to that of transducer cage 12.

Tube portion 110 is a flexible cylindrical tube, ideally having an inner diameter slightly larger than the radius of sensor cable 20, and about equal to that of opening 124 in body tip 120. Tube portion 110 should be sufficiently flexible to permit a range of motion about equal to that permitted by sensor cable 20. Also, advantageously, tube portion 110 should have sufficient columnar stiffness to maintain its shape without the aid of an additional support structure, such as a rod disposed within the tube portion.

A tube having both the desired flexibility and rigidity can advantageously be manufactured in an extrusion process wherein a first layer of fluoropolymer such as FEP is extruded onto a rigid mandrel which may be a copper wire coated with a plastic material such as Celcon® manufactured by Celanese Chemicals of Europe, GmBH. The FEP is subsequently coated with one or more layers of polyurethane, which may subsequently be cured, such as by heat, as is well known in the art. Other methods of manufacturing such a tube will be immediately recognized and well known to a person of skill in the art. Preferably, the structure of the laminated tubing from which tube portion 110 is formed is reinforced by a fiber winding such as an aramid fiber which is encapsulated by one or more additional layers of polyurethane. Alternately, a fibrous braid or a metal wire may also be advantageously be used to reinforce the tube portion. Using this method, tubing having the desired characteristics for use in probe body 100, particularly longitudinal flexibility and columnar stiffness, can be produced continuously, removed from the mandrel and cut to an appropriate length for use in the present invention. Other methods for producing the tubing herein described, as well as other types of tubing having similar longitudinal flexibility and columnar stiffness will be known to a person of skill in the art and can be substituted for the method and material disclosed herein with similar results. Markings (not shown) may also be provided on the outer surface of tube portion 110 at regular intervals to visibly indicate one or more predetermined distances from the distal end of the body.

As discussed above, opening 124 of body tip 120 is connected to the distal end of tube portion 110, and sealed thereto, such as by heat. The seal between body tip 120 and tube portion 110 of probe body 100 must be complete to prevent the movement of fluids or contaminants between the outside and the inside of probe body 110.

Hub 130 is a rigid cylindrical tube, preferably formed of plastic such as Isoplast® manufactured by Dow Chemical Co. of Midland Mich., having a flange 132 at a distal end thereof with a bore diameter approximately equal to the outer diameter of tube portion 110. Connector 134 is located at a proximal end of hub 130 and is provided with interlocking slot 136 having recess 137 provided therein. Hub 130 is attached to the proximal end of tube portion 110 at flange 132, such as by insert molding. The seal between flange 132 and tube portion 110 is ideally sufficiently complete to prevent the exchange of fluids or contaminants across the seal. Longitudinal dimension Lp of probe body 100 is ideally about equal to the length of sensor cable 20.

The assembly of probe body 100 onto probe 10 can be accomplished simply by inserting the distal end of sensor cable 20, particularly transducer cage 12, into the proximal end of probe body 100. Specifically, sensor cable 120 should extend into tube portion 110 nearly completely, resulting in the placement of transducer cage 12 within body tip 120. The self-supporting structure of probe body 100 allows for the escape of air during installation onto probe 10 between sensor cable 20 and the probe body 100 because the semi-rigid structure of the probe body does not form a good seal against the surface of the sensor cable, leaving small gaps through which air can pass.

Hub 130 is adapted to be received within hub receptacle 60 in body base 42, annular shoulder 62 having a bore sufficient to accommodate connector 134 and interlocking slot 136 receiving the portion of guide pin 64 that extends into annular shoulder 62. This removable “bayonet style” connector can be locked after insertion by rotation of hub 130 relative to body base 42, thereby locking guide pin 64 within recess 137. Reversal of this process unlocks connector 134, allowing hub 130 to be removed. Other removable connector designs will be immediately known to one of skill in the art and may be substituted for the connector arrangement herein disclosed. The interface between hub 130 and hub receptacle 60 need not form a microbial seal, although such a seal may be provided where an o-ring (not shown), or similar seal, is added between connector 134 and annular shoulder 62.

When probe body 100 is installed over sensor cable 20 and locked into body base 42, circuitry in optical sensor assembly 70 detects the presence of the locked hub 130 and indicates, such as visually or audibly, that probe 10 is microbially isolated and is ready for use. Optical sensor assembly 70 may optionally lock out the use of the sensor, for example by blocking the transmission of transducer signals or by otherwise providing a signal which prevents the probe from functioning, if probe body 100 is not properly installed. This ensures that an uncovered probe is never used on a patient.

In operation, probe 10 may be inserted into a body cavity, such as the esophagus, of a patient for the purpose, for example, of obtaining cardiac telemetry. When the probe has been inserted to the appropriate depth, which process may be facilitated by measured markings on probe body 100, sensor cable 20 can be rotated to position sensing elements 14 into proper alignment by rotating sensor base handle 80 relative to body base 42, causing sensor cable 20 to rotate within probe body 100. As noted above, the margin for error in alignment is relatively narrow, therefore, any translational loss, or backlash in the sensor cable is likely to have a significant adverse impact on the acquisition of data by probe 10. Due to the angular rigidity of sensor cable 20, rotation of sensor base handle 80 is directly translated along the length of sensor cable 20 at a ratio of approximately 1:1. Therefore, there is no appreciable backlash due to cable distortion which might affect alignment of sensing elements 14. Furthermore, the angular rigidity of sensor cable 20 prevents the development of stored energy in the form of unresolved twisting in the in the cable which could resolve later, upsetting the alignment unexpectedly.

Under normal use, an interface cable attached to connectors 88 connects probe 10 to a data processor/display screen. Rotational stop assembly 50 allows the alignment of the sensor elements 14 to take place without the risk that the interface cable will be damaged by excessive rotation of sensor base handle 80 in one direction. A range of rotation, such as 540 degrees, ensures that proper alignment is possible from any insertion orientation without the risk of damage to the interface cable.

When probe 10 is removed from a patient, probe body 100 may be removed from sensor cable 20 and discarded. Optical sensor assembly 70 detects the removal of hub 130 from hub receptacle 60 and indicates, either audibly or visually that probe 10 is not ready for use. Optionally, optical sensor assembly 70 may interrupt transmission of signals to/from sensing elements 14 until a new probe body 100 is installed. When a new probe body is installed, probe 10 is again ready for use, without the need for thorough sterilization or disinfection of the probe between uses.

An impedance-matching medium, such as an ultrasonic gel is typically needed to displace air between transducer cage 12 and the inner surface of body tip 120. The gel is preferably loaded in body tip 120 prior to installation on probe 10, and must be kept in place while probe body 100 is in storage. Additionally, despite the columnar stiffness demonstrated by tube portion 110, it may still be desirable to store new probe bodies with a support to protect them from damage before use.

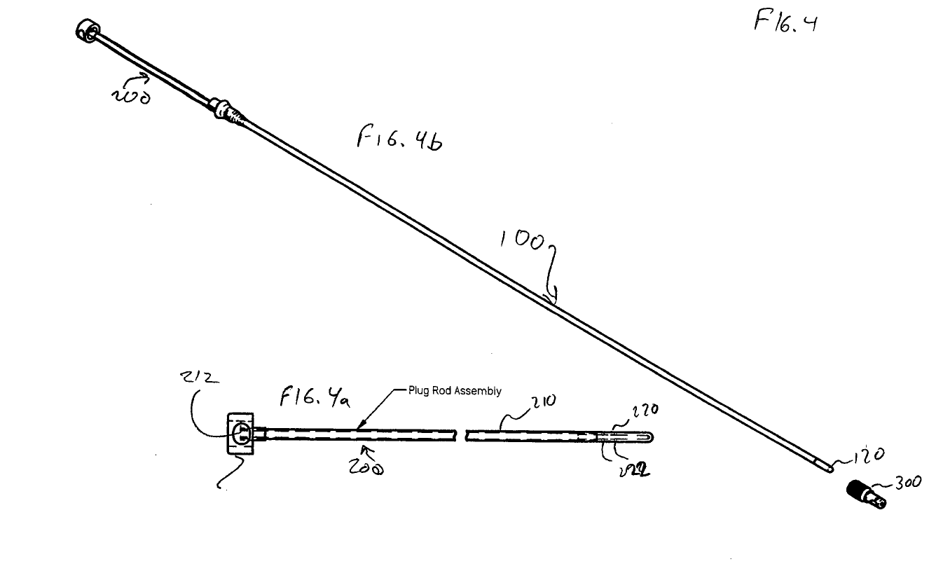

As shown in FIGS. 4a and 4b, a plug rod assembly 200 may be provided to lock an impedance matching material, such as an ultrasonic gel into place within body tip 120, and also to support and protect probe body 100 prior to use. Plug rod assembly 200 comprises a flexible tubular rod 210, having a longitudinal dimension ideally slightly less than that of probe body 100 and a hollow passage 212 therein which is in fluid communication with a distal tip 220. Distal tip 220 has air holes 222 disposed therein which extend from the surface of distal tip 220 to establish fluid communication with hollow passage 212. A handle 230 is provided at a proximal end of tubular rod 210 and is secured thereto.

FIG. 4b shows plug rod assembly 200 partially inserted within probe body 100. As plug rod assembly 200 is inserted into probe body 100, any air trapped probe body 100 escapes through air holes 222 and exits from the proximal end of tubular rod 210 via hollow passage 212. When fully inserted, distal tip 220 comes to rest against a reservoir of ultrasonic gel (not shown) preventing it from migrating out of body tip 120. Additionally, a tip cap, 300 may be provided to compress the proximal end of body tip 120 against plug rod assembly 200, further restricting the proximal migration of ultrasonic gel. Furthermore, tubular rod 210 functions to support the structure of probe body 100, protecting it from damage prior to use.

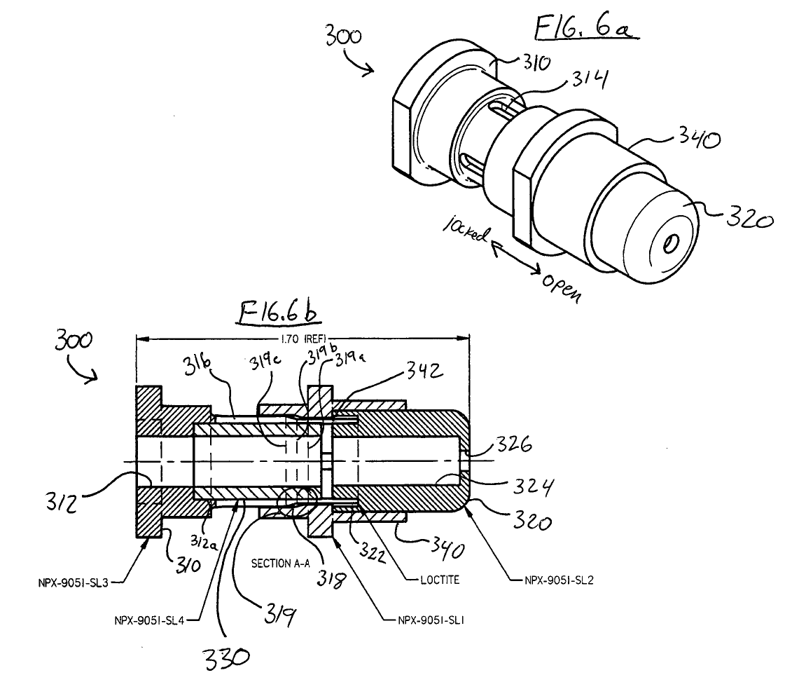

FIGS. 6a and 6b show an embodiment of a tip cap 300. Specifically, clamping flange 310 is shown at a proximal end, and tip housing 320 is shown at a distal end thereof. Clamping flange 310 is generally cylindrical and has a coaxial cylindrical bore 312 therein, the diameter of which is ideally slightly greater than the outer diameter of probe body 100 at the proximal end of clamping flange 310. Tip housing 320 is also cylindrical, having a cylindrical bore 324 which extends from a proximal end thereof and terminates at a distal end and may have a diameter approximately equal to cylindrical bore 312 in clamping flange 310. Ideally, the diameter of bore 324 exceeds the outer diameter of body tip 120 just enough to allow body tip 120 to enter easily into bore 324. Cylindrical bore 324 has a longitudinal dimension approximately equal to that of body tip 120. Cylindrical bore 324 defines a proximal opening in tip housing 320 and hole 326 in tip housing 320 provides an distal opening therein. The distal end of clamping flange 310 is received within annular groove 322 in tip housing 320. Preferably, the clamping flange and the tip housing are made of rigid plastic material, and fastened to each other at annular groove 322 by means such as adhesive. Alternately, clamping flange 310 and tip housing 320 may be attached together advantageously by a bond such as an ultrasonic weld which avoids the use of adhesive.

A series of longitudinal slots 314 define a plurality of arcuate tines 316 which extend from clamping flange 310 to its distal end. The diameter of bore 312 increases at 312a to accommodate a resilient sleeve 330, which has an outer diameter approximately equal to that of bore 312a and an inner diameter approximately equal to that of bore 312, and is preferably formed from a resilient material such as natural or synthetic rubber. Each of said arcuate tines is 316 has a chamfer 318 which defines a circumferential taper 319 defined by an outer diameter which increases from 319a-319c moving from the distal to the proximal end of the taper.

A locking ring 340 is slidably mounted about clamping flange 310 having a generally annular shape and having a reduced inner diameter 342 which is, at least in part, approximately equal to diameter 319a, and less than diameter 319c. Ideally, locking ring 340 is mounted for sliding between a distal open position and a proximal, locked position. When locking ring 340 is in the open position, reduced inner diameter 342 is located over a portion of clamping flange 310 having outer diameter 319a. Conversely, when locking ring 340 is in the locked position, reduced diameter 342 is disposed over a portion of clamping flange 310 having outer diameter 319c.

In use, the tip cap 300 is placed over the body tip 120 of probe body 100. Bores 312 and 324 admit body tip 120 and part of tube portion 110 of probe body 100. Ideally, the distal end of tube portion 110 is located within resilient sleeve 330 when probe body 100 is fully inserted within tip cap 300. Due to the sizing of bore 312, probe body 100 moves easily within tip cap 300 when locking ring 300 is in the open position. However, when locking ring 340 is moved into the locked position, reduced inner diameter 342 is forced over a portion of clamping flange 310 which has a larger outer diameter than that of 342. Therefore, arucate tines 316 are forced to flex radially inward, causing resilient sleeve 330 to seal against tube portion 110 of probe body 100. This action locks the tube portion 110 in place against plug rod 200, further preventing the migration of acoustic gel proximally from body tip 120.

The tip cap of the present embodiment is particularly advantageous because the force exerted by arcuate tines 316 upon probe body 100 is distributed evenly, and across a wider surface area by resilient sleeve 330. This prevents pressure points from forming in tube portion 110 which may cause damage thereto and ensures a complete seal between the tube portion 110 and plug rod 200 about the entire circumference thereof. Further, the ability to lock tip cap 300 after insertion of probe body 100 prevents the unintended compression of body tip 120 which may cause gel to exit body tip 120.

A further advantage of the tip cap of the present embodiment is realized during sterilization of the probe body 100. Preferably, the outer surface of probe body 100 requires sterilization prior to use. Because any surface that contacts probe body 100 must therefore also be sterile, tip cap 300 and probe body 100 are preferably sterilized together in the same operation. A preferred method of sterilization involves the placement of the probe body 100 into a hermetically sealed chamber, subsequently evacuating the chamber, and then filling the chamber with a sterilizing gas. This process, particularly the evacuation step, causes expansion of the body tip 120, particularly when the interior of probe body 100 is exposed to atmospheric pressure. During this phase, expansion of the body tip 120, which might otherwise result in damage or rupture, is restricted by bore 324 which is advantageously only slightly larger than the unexpanded diameter of body tip 120. A further advantage is realized by hole 326, which allows the egress of air during insertion of probe body 100, and also permits the ingress of sterilizing gasses during the sterilization process.

When needed, plug rod assembly 200 may be removed from probe body 100 by pulling. As tubular rod 210 is withdrawn from probe body 100, vacuum created within probe body 100 is relieved by air which enters the probe body via hollow passage 212. This avoids disturbance, or withdrawal of excessive gel material during removal of the plug rod assembly 200. When the plug rod assembly is fully withdrawn, probe body 100 is ready for use.

In another alternate embodiment of the present invention, body tip 120 is provided with an internal surface that reduces the appearance of unwanted “echo signals” which can adversely affect the acquisition of data using probe 10, particularly cardiac telemetry.

In practice, ultrasonic signals will not only reflect from a desired target to provide meaningful data, but that the same signal will also be reflected from any object or surface which lies between the source of an ultrasonic signal and its target. The data reflected from these intermediate surfaces is not meaningful and, when coherent, can often be mistaken during analysis by processing software, or by a human being for the target reflection, leading to erroneous results. It is therefore desirable to minimize the occurrence of spurious signals, particularly those which are coherent and therefore may mimic the waveform of the target signal.

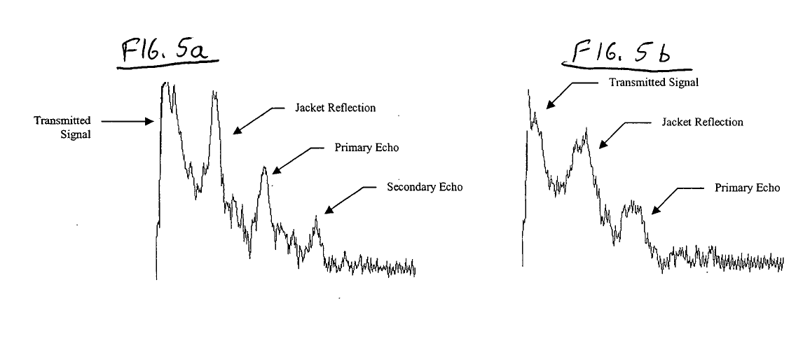

A further embodiment of the present invention seeks to reduce the coherence and signal strength of spurious signals, thereby facilitating the identification of the target signal. This is accomplished by modifying the interior surface of body tip 120 at the distal end of probe body 100. FIGS. 5a and 5b illustrate a comparison between a typical body tip 120 formed by dip casting a plastic material such as Texin® and having a smooth internal surface (FIG. 5a) and an alternate embodiment formed of injection molded Texin® and having a longitudinally ribbed surface (FIG. 5b).

As shown in the graphs the primary echo, which is the target signal, is significantly reduced in amplitude as a result of employing a ribbed body tip surface in FIG. 5b compared to the corresponding primary echo shown in FIG. 5a. However, spurious signals due to jacket reflection and secondary echo are also significantly reduced using the ribbed body tip surface. Particularly the secondary echo, which is most likely to be mistaken for the primary echo, is practically eliminated in FIG. 5b. Thus, despite an overall reduction in the signal strength of the target signal as a result of employing a ribbed surface, the elimination of the secondary echo in FIG. 5b represents an overall increase in signal quality.

While the present invention has been illustrated in some detail according to the preferred embodiment shown in the foregoing drawings and description, it will become apparent to those skilled in the art that variations and equivalents may be made within the spirit and scope of that which has been expressly disclosed. Accordingly, it is intended that the scope of the invention be limited solely by the scope of the hereafter appended claims and not by any specific wording in the foregoing description.

1. An intracorporeal probe for use within a human body, comprising:
a flexible, axially elongated, rotatable sensor cable mounted at a proximal end to a sensor base and having at least one ultrasound transducer mounted on a distal portion thereof;
a disposable, elongated flexible body having a tubular configuration, an open proximal end and a closed distal end, said body being adapted, configured and dimensioned to slidably receive said sensor cable therein without collapse or kinking; and
a proximal hub attached to said flexible body for removably securing said flexible body to a body base to microbially isolate said sensor cable and wherein said sensor base is configured to rotate relative to said body base, causing said sensor cable to rotate relative to said flexible body.

2. The intracorporeal probe of claim 1 wherein said distal end of said body comprises a sonolucent sensor window containing an acoustic gel therein, and wherein said at least one transducer is configured to face outwardly through said sensor window.

3. The intracorporeal probe of claim 2 wherein said sensor window is formed of a transparent material to permit said at least one transducer to be visually inspected through said flexible body.

4. The intracorporeal probe of claim 2 wherein said sensor window is provided with an anechoic surface treatment.

5. The intracorporeal probe of claim 4 wherein said anechoic surface treatment comprises a plurality of longitudinal ribs disposed in said sensor window.

6. The intracorporeal probe of claim 1 wherein said body base further comprises a detector assembly which detects the presence of said hub to confirm securement of said flexible body to said body base.

7. The intracorporeal probe of claim 6 wherein said detector assembly is electrically associated with said sensor base and provides a visual indication on the probe that said flexible body is secure.

8. The intracorporeal probe of claim 6 wherein said detector assembly is electrically associated with said at least one transducer and prevents the probe from functioning when a hub is not detected.

9. The intracorporeal probe of claim 1 wherein said body base and said sensor base are concentrically nested and said sensor base is configured for limited rotational movement relative to said body base.

10. The intracorporeal probe of claim 9 wherein said limited rotational movement of said sensor base relative to said body base is governed by a rotational stop assembly means.

11. The intracorporeal probe of claim 2 wherein said at least one transducer is housed within a rigid transducer cage, said cage having at least one groove provided therein to facilitate the flow of said gel about said at least one transducer

12. The intracorporeal probe of claim 1 wherein said sensor cable is comprised of a plurality of metallic windings defining a coaxial channel therein, said windings configured to resist twisting distortion due to rotation of the sensor cable within said flexible body.

13. The intracorporeal probe of claim 12 wherein said metallic windings are comprised of medical grade stainless steel wire.

14. The intracorporeal probe of claim 12 wherein said metallic windings are coated in polyurethane.

15. The intracorporeal probe of claim 12 further comprising at least one transducer conductor electrically associated with said at least one transducer, said transducer conductor extending axially within said coaxial channel.

16. The intracorporeal probe of claim 15 further comprising a connector at a proximal end of said sensor base, said connector electrically associated with said transducer conductor for providing a terminus for connection to said at least one transducer.

17. The intracorporeal probe of claim 12 wherein at least one Kevlar strand is provided among said metallic windings for increasing the tensile strength thereof.

18. A flexible body for protecting the sensor cable of an intracorporeal probe, comprising:
a flexible fiber reinforced hollow tube having a proximal open end and a distal closed end, said closed end formed of a sonolucent material and containing an acoustic gel therein, said tube being adapted, configured and dimensioned to slidably receive therein without collapse or kinking said sensor cable; and
a proximal hub connector secured to said open end and defining means for securing said open end and a distal end of a body base.

19. The device of claim 18 further comprising a protective tip removably disposed on said distal closed end to protect said closed end and limit leakage of acoustic gel therefrom.

20. The device of claim 19 further comprising a plug rod removably partially disposed in said flexible body, said plug rod having a proximal end configured dimensioned for releasably engaging said open end and a distal end configured and dimensioned for maintaining the acoustic gel within said closed end.

21. The device of claim 19 wherein said protective tip comprises a selectively compressible resilient sleeve which engages said flexible body proximal said distal closed end, wherein said resilient sleeve seals against said body when compressed for maintaining the acoustic gel distally of said resilient sleeve.

22. The device of claim 21 wherein at least a portion of said protective tip defines a bore having a radial dimension sufficiently large to admit said distal closed end of said flexible body, and sufficiently small to prevent rupture of said closed distal end due to air pressure.

23. The device of claim 22 wherein the distal end of said protective tip has an opening defining a hole in fluid communication with said bore, and allowing passage of gas therethrough.
